(19) United States
(12) Patent Application Publication
(10) Pub. No.: US 2026/0232758 A1
(43) Pub. Date: Aug. 13, 2026

(54) Low Cost Efficient Intracellular Delivery of Proteins and Nucleic Acids via Hydrophobic Ion Pairing

(71) Applicant: Georgia Tech Research Corporation, Atlanta, GA (US)

(72) Inventors: Julie A. Champion, Atlanta, GA (US); Mikaela A. Gray, Atlanta, GA (US)

(21) Appl. No.: 19/160,353

(22) PCT Filed: Mar. 1, 2024
(86) PCT No.: PCT/US2024/018138

§ 371 Date: Aug. 27, 2025

Related U.S. Application Data
(60) Provisional application No. 63/487,701, filed on Mar. 1, 2023.
(60) Provisional application No. 63/547,372, filed on Nov. 5, 2023.

(51) Int. Cl. A61K 38/16; A61K 31/7105; A61K 31/711; A61K 47/12; A61K 47/18; A61K 47/20

(52) U.S. Cl. CPC A61K 38/16; A61K 31/7105; A61K 31/711; A61K 47/12; A61K 47/18; A61K 47/20

(57) ABSTRACT

An exemplary embodiment of the present disclosure provides an intracellular delivery complex, comprising at least one biomacromolecule, each of the at least one biomacromolecules having a first ionic net charge, and at least one hydrophobic counterion, each of the at least one hydrophobic counterions having a second ionic net charge opposite the first ionic net charge, and wherein the complex is not encapsulated in a nanocarrier. Another embodiment provides a method of delivering a biomacromolecule into a cell, which comprises providing a biomacromolecule as described herein, a hydrophobic counterion as described herein, and mixing the two under conditions effective to form a complex capable of entering a cell. Another embodiment provides a method of administering a biomacromolecule into a cell, which comprises providing the intracellular delivery complex of the present disclosure, and administering the intracellular delivery complex to a subject, such that the complex enters a cell of the subject.

### CROSS-REFERENCE TO RELATED APPLICATIONS

This application claims the benefit of U.S. Provisional Application Ser. No. 63/487,701, filed on Mar. 1, 2023, and U.S. Provisional Application Ser. No. 63/547,372, filed on Nov. 5, 2023, which are incorporated herein by reference in their entirety as if fully set forth below.

### GOVERNMENT LICENSE RIGHTS

This invention was made with government support under Award No. 2104734, awarded by the National Science Foundation. The government has certain rights in the invention.

### FIELD OF THE DISCLOSURE

The various embodiments of the present disclosure relate generally to low cost, efficient intracellular delivery of proteins and nucleic acids via hydrophobic ion pairing.

### BACKGROUND

Transfection of cells with nucleic acids, such as deoxyribonucleic acids (DNAs) or ribonucleic acids (RNAs), or proteins is a critical step in research and therapeutics. The most common methods of transfection are biological (ex. viruses), chemical (ex. nanoparticles), and physical (ex. electroporation). Chemical methods face challenges of endosomal entrapment and degradation. One commonly used cationic lipid transfection agent, lipofectamine, complexes with negatively charged nucleic acids to overcome the electrostatic repulsion from the cell membrane by using a neutral helper co-lipid to increase transfection efficiency. Lipofectamine however, has undesired toxicity, which is positively correlated to lipofection agent transfection efficiency. Thus, a variety of nanocarriers have been utilized to improve biomacromolecule delivery.

Hydrophobic ion pairing (HIP) improves encapsulation efficiency in nanoparticles by pairing a charged hydrophilic biomacromolecule with an oppositely charged hydrophobic ion. HIP relies on ionic interactions between a charged hydrophilic biomacromolecule of interest with an oppositely charged counterion containing one or many hydrophobic domains. This method temporarily increases the hydrophobicity of a biomacromolecule by binding hydrophobic domains and masking the biomacromolecule's natural charge. A simulation of HIP complex formation demonstrated that electrostatic interactions between the ion and biomacromolecule drive cluster growth, then the clusters merge over time where the biomacromolecule decorates the complex surface and the counterion's hydrophobic areas are sequestered into the center of the cluster. The ratio of biomacromolecule to counterion determines the rate of biomacromolecule release where more counterion results in slower release. HIP complexes encapsulated in various nanoparticle delivery systems have been shown to improve membrane interaction from their high membrane solubility and transport capabilities. Additionally, HIP complexes in self-emulsifying drug delivery systems; composed of drug with oil, surfactant, and cosurfactant, have enhanced microbial antibiotic effectiveness from binding to calcium and magnesium to destabilize membranes and increase delivery efficiency. Intracellular delivery using HIP has been investigated using a cationic lipid, DOTAP, along with siRNA whereby prolonged release occurred over 10-13 days in solid lipid nanoparticles.

The present disclosure is directed to overcoming limitations in the art.

### BRIEF SUMMARY

An exemplary embodiment of the present disclosure provides an intracellular delivery complex, comprising at least one biomacromolecule, each of the at least one biomacromolecules having a first ionic net charge, and at least one hydrophobic counterion, each of the at least one hydrophobic counterions having a second ionic net charge opposite the first ionic net charge, and wherein the complex is not encapsulated in a nanocarrier.

In any of the embodiments disclosed herein, the at least one hydrophobic counterions comprises one charge group and no hydrophilic head group.

In any of the embodiments disclosed herein, the at least one biomacromolecules is a protein, peptide, or nucleic acid.

In any of the embodiments disclosed herein, the at least one hydrophobic counterions is a cation.

In any of the embodiments disclosed herein, the at least one hydrophobic counterions is an anion.

In any of the embodiments disclosed herein, the at least one hydrophobic counterions is selected from the group consisting of oleic acid, sodium docusate, benethamine, arginine hexadecanoyl ester, arginine-nonyl ester, lauramide arginine ethyl ester, 2-naphthalene sulfonic acid, carboxy methyl polyethylene glycol, cholesterol hemisuccinate, decanoic acid, docosahexaenoic acid, linoleic acid, sodium acetate, vitamin E succinate, dodecylamine, maprotiline, N,N-dimethyl dodecylamine, N,N-dimethyl hexylamine, N,N-dimethyl octadecylamine, stearylamine, tetrabutyl ammonium bromide, and triethylamine.

In any of the embodiments disclosed herein, a molar ratio of the biomacromolecules to the hydrophobic counterions in the complex is about 1:10⁻¹⁰ to about 1:396.

In any of the embodiments disclosed herein, the at least one biomacromolecule and the at least one hydrophobic counterion are present in a charge ratio of about 1:10⁻¹¹ to about 1:44.

In any of the embodiments disclosed herein, the at least one biomacromolecule and the at least one hydrophobic counterion are present in a mass ratio of about 1:10⁻¹² to about 1:15.

In any of the embodiments disclosed herein, the at least one biomacromolecule comprises a molecule of cytochrome C and the at least one hydrophobic counterion comprises an oleic acid ion.

In any of the embodiments disclosed herein, the at least one biomacromolecule comprises a molecule of cytochrome C and the at least one hydrophobic counterion comprises a sodium docusate ion.

In any of the embodiments disclosed herein, the at least one biomacromolecule comprises a molecule of siRNA and the at least one hydrophobic counterion comprises a benethamine ion.

In any of the embodiments disclosed herein, the at least one biomacromolecule comprises a molecule of plasmid DNA and the at least one hydrophobic counterion comprises a benethamine ion.

In any of the embodiments disclosed herein, the first ionic net charge has a first magnitude and the second ionic net charge has a second magnitude different than the first magnitude.

Another embodiment of the present disclosure provides a method of delivering a biomacromolecule into a cell. This method comprises providing at least one biomacromolecule, each of the at least one biomacromolecules having a first ionic net charge, providing at least one hydrophobic counterion, each of the at least one hydrophobic counterions having a second ionic net charge opposite the first ionic net charge, mixing the at least one biomacromolecule with the at least one hydrophobic counterion under conditions effective to form a complex capable of entering a cell, wherein the complex is not encapsulated in a nanocarrier.

In any of the embodiments disclosed herein, the complex is capable of entering a cell via endocytosis.

In any of the embodiments disclosed herein, the method can further comprise administering the complex to a subject such that the complex enters a cell via endocytosis.

In any of the embodiments disclosed herein, the mixing can be carried out in the presence of an aqueous buffer.

Another embodiment of the present disclosure provides a method of administering a biomacromolecule into a cell. This method comprises providing an intracellular delivery complex, comprising at least one biomacromolecule, each of the at least one biomacromolecules having a first ionic net charge; and providing at least one hydrophobic counterion, each of the at least one hydrophobic counterions having a second ionic net charge opposite the first ionic net charge, and wherein the complex is not encapsulated in a nanocarrier; and administering the intracellular delivery complex to a subject, such that the complex enters a cell of the subject.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF THE DRAWINGS

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIGS. 1A-1C provide counterion structures of oleic acid (OA) (FIG. 1A) with a Log P=6.78, sodium docusate (SD) (FIG. 1B) with a Log P=5.2, and benethamine (FIG. 1C) with a Log P=3.6.

FIGS. 2A-2C provide surface potential of Cytochrome C (FIG. 2A), sfGFP(−10) (FIG. 2B), and sfGFP(+10) (FIG. 2C) per amino acid determined by using the protein sequence to predict structure, electrostatics calculations, and surface analysis (Hebditch and Warwicker, “Web-Based Display of Protein Surface and PH-Dependent Properties for Assessing the Developability of Biotherapeutics,” Scientific Reports, 9 (1): 1969 (2019); Mirdita et al., “ColabFold: Making Protein Folding Accessible to All,” Nature Methods, 19 (6): 679-682 (2022); Dolinsky et al., “PDB2PQR: An Automated Pipeline for the Setup of Poisson-Boltzmann Electrostatics Calculations,” Nucleic Acids Res, 32 (Web Server issue): W665-667 (2004)). FIGS. 2D-2E provide gels of sfGFP(+10) (FIG. 2D) and sfGFP(−10) (FIG. 2E) purified using nickel affinity similar to inventors' previous work (Dautel and Champion, “Protein Vesicles Self-Assembled from Functional Globular Proteins with Different Charge and Size,” Biomacromolecules, 22 (1): 116-125 (2021)).

FIGS. 3A-3E provide the influence of HIP on protein structure of model proteins sfGFP(+10), sfGFP(−10), and Cytochrome C with their respective counterions: oleic acid and sodium docusate, or benethamine, in accordance with some embodiments of the present disclosure, as measured by loss in protein absorbance or fluorescence indicating loss in concentration of folded protein. FIG. 3A shows the influence of oleic acid and sodium docusate on concentration of folded Cytochrome C. FIG. 3B shows the influence of oleic acid on concentration of folded sfGFP(+10). FIG. 3C shows the influence of sodium docusate on concentration of folded sfGFP(+10) to sodium docusate. FIG. 3D shows the influence of benethamine on concentration of folded sfGFP(−10). 2× HIP sfGFP(+10) SD indicates a charge ratio two times that used in the delivery experiments equal to 1:10.8. Only counterions which yielded less than 30% reduction in folded protein were used. FIG. 3E shows the influence of trimethyl chitosan on concentration of folded sfGFP(−10).

FIGS. 4A-4D provide CytC HIP complex characterization and cytosolic delivery in adherent and suspension cell types, in accordance with some embodiments of the present disclosure. FIG. 4A shows TEM micrographs to visualize HIP complex structures. HIP groups were composed of equal volumes of 50 μL 1 mg/ml CytC with 50 μL 3 mg/ml of OA or 4 mg/ml of SD. 50 μL CytC was mixed with an equal volume of 1×PBS because counterions were dissolved in 1×PBS. All groups were characterized without prolonged incubation. FIG. 4B shows quantification of CytC HIP complex size with different counterions. Samples were measured in triplicates where a z-average hydrodynamic diameter distribution was calculated based on sample intensity. FIG. 4C shows quantification of influence on HeLa cellular metabolism using 3-(4,5-dimethylthiazol-2-yl)-2,5-diphenyltetrazolium bromide (MTT) assay. 20 μM CytC and 6 μM Dox were treated to 15,000 cells/well in a 96 well plate. Influence on cell viability after 48 hours was normalized to PBS treated cell viability. All groups were treated in the presence of serum containing media. FIG. 4D shows quantification of influence on K562 cellular metabolism using 3-(4,5-dimethylthiazol-2-yl)-2,5-diphenyltetrazolium bromide (MTT) assay. 20 μM CytC was treated to 15,000 cells/well in a 96 well plate. Influence on cell viability after 48 hours was normalized to PBS treated cell viability. All groups were treated in the presence of serum containing media.

FIGS. 5A-5D provide changes in HIP complex structures at charge ratios of 1 CytC: 1.8 OA (FIG. 5A), 1 CytC: 5.3 OA (FIG. 5B), and 1 CytC: 16 OA (FIG. 5C), and influence on cell viability in HeLa cells after 48 hours as charge ratio of OA or SD increases within a hydrophobic complex with 20 μM Cytochrome C (FIG. 5D), in accordance with some embodiments of the present disclosure. Statistical significance begins at a charge ratio of 10.66 for both counterions.

FIGS. 6A-6C provide HIP complex route of uptake and mechanism of cytosolic delivery, in accordance with some embodiments of the present disclosure. FIG. 6A shows hemolytic activity of sfGFP(−10), BA, and HIP sfGFP(−10) BA at 37° C. FIG. 6B shows energy dependent endocytosis uptake and FIG. 6C shows that inhibiting ATP processes and neutralizing membrane potential using sodium azide to chemically inhibit endocytosis proves HIP complexes are dependent on endocytosis for uptake (Mckinlay et al., “Cell-Penetrating, Guanidinium-Rich Oligophosphoesters: Effective and Versatile Molecular Transporters for Drug and Probe Delivery,” J. Am. Chem. Soc., 138 (10): 3510-3517 (2016)).

FIG. 7 provides hemolytic activity for Cytochrome C, OA, SD, HIP CytC OA, and HIP CytC SD at 37° C. and 4° C. to show the influence of membrane stiffness on membrane interaction, in accordance with some embodiments of the present disclosure. 20 μM CytC was given to each group and the concentration of counterion given was consistent with the charge ratios used for delivery experiments. Washed red blood cells were diluted to 0.5% v/v in PBS and 1% v/v Triton X-100 was used as the positive control.

FIGS. 8A-8E provide sfGFP(+10) HIP complex characterization, uptake, and influence on cell viability, in accordance with some embodiments of the present disclosure. FIG. 8A shows epifluorescent micrographs to visualize HIP complex structures. HIP groups were composed of equal volumes of 50 μL 1 mg/ml sfGFP(+10) with 50 μL 4 mg/ml of OA or 1 mg/ml of SD. 50 μL sfGFP(+10) was mixed with an equal volume of 1×PBS because counterions were dissolved in 1×PBS. All groups were characterized without prolonged incubation. FIG. 8B shows quantification of sfGFP(+10) HIP complex size with different counterions. Samples were measured in triplicates where a z-average hydrodynamic diameter distribution was calculated based on sample intensity. FIG. 8C shows quantification of sfGFP(+10) HIP complex uptake by live HeLa cells after 24 hours. 1 μM sfGFP(+10) was given in the presence of FBS for all groups to 15,000 cells/well in a 96 well plate. sfGFP(+10) was mixed with counterion then mixed with FBS containing media and given to cells. Live cell populations were selected after adding trypan blue to trypsinized cells using flow cytometry. Median fluorescein isothiocyanate (FITC) area, plotted as relative fluorescence units (R.F.U.) was utilized to compare uptake. FIG. 8D shows quantification of influence on cellular metabolism using 3-(4,5-dimethylthiazol-2-yl)-2,5-diphenyltetrazolium bromide (MTT) assay. 1 μM sfGFP(+10) was given in the presence of FBS for all groups to 15,000 cells/well in a 96 well plate. sfGFP(+10) was mixed with counterion then mixed with FBS containing media and given to cells. Influence on cell viability after 24 hours was normalized to PBS treated cell viability. FIG. 8E shows quantification of RBC HIP extracellular binding at pH 7.4 and pH 5. 0.5% washed RBC were treated for 1 hour with 1 μM sfGFP(+10), washed, and analyzed using FITC signal from flow cytometry.

FIGS. 9A-9E provide sfGFP(−10) HIP complex characterization, uptake, and influence on cell viability, in accordance with some embodiments of the present disclosure. FIG. 9A shows a TEM micrograph of HIP complex to visualize HIP complex structures. HIP groups were composed of equal volumes of 50 μL 1 mg/ml sfGFP(−10) with 50 μL 1 mg/ml of BA. 50 μL sfGFP(−10) was mixed with an equal volume of 1×PBS because counterions were dissolved in 1×PBS. All groups were characterized without prolonged incubation. FIG. 9B shows quantification of sfGFP(−10) HIP complex size with different counterions. Samples were measured in triplicates where a z-average hydrodynamic diameter distribution was calculated based on sample intensity. FIG. 9C shows quantification of sfGFP(−10) HIP complex uptake by live HeLa cells after 24 hours. 1 μM sfGFP(−10) was given in the presence of FBS for all groups to 15,000 cells/well in a 96 well plate. sfGFP(−10) was mixed with counterion then mixed with FBS containing media and given to cells. Live cell populations were selected after adding trypan blue to trypsinized cells using flow cytometry. Median fluorescein isothiocyanate (FITC) area, plotted as relative fluorescence units (R.F.U.) was utilized to compare uptake. FIG. 9D shows quantification of influence on cellular metabolism using 3-(4,5-dimethylthiazol-2-yl)-2,5-diphenyltetrazolium bromide (MTT) assay. 1 μM sfGFP(−10) was given in the presence of FBS for all groups to 15,000 cells/well in a 96 well plate. sfGFP(−10) was mixed with counterion then mixed with FBS containing media and given to cells. Influence on cell viability after 24 hours was normalized to PBS treated cell viability. FIG. 9E shows quantification of RBC HIP extracellular binding at pH 7.4 and pH 5. 0.5% washed RBC were treated for 1 hour with 1 μM sfGFP(−10), washed, and analyzed using FITC signal from flow cytometry.

FIGS. 10A-10D provide siRNA HIP complex characterization and GFP knockdown, in accordance with some embodiments of the present disclosure. FIG. 10A shows measurement of HIP complex hydrophobicity using the Log D partition coefficient. HIP complexes and siRNA at different time points (day 1-5) were resuspended into an equal volume of butanol and water and mixed with 150 rpm overnight. The nucleic acid concentration in each phase was calculated using A260 where Log D is the natural logarithm of the concentration in butanol divided by the concentration in water. Positive values indicate more hydrophobic while negative values indicate more hydrophilic. Day 3 produced the first ratio which yielded a significantly different hydrophobicity than uncomplexed siRNA. FIG. 10B shows quantification of siRNA HIP complex size with BA. HIP groups were composed of equal volumes of 50 μM siRNA with 4 mg/ml of BA. siRNA was mixed with an equal volume of 1×PBS because counterions were dissolved in 1×PBS. All groups were characterized without prolonged incubation. Samples were measured in triplicates where a z-average hydrodynamic diameter distribution was calculated based on sample intensity. FIG. 10C shows quantification of siRNA BA HIP complex GFP expression in NIH/eGFP3T3 cells after 48 hours. 75 nM siRNA was given in the presence of FBS for all groups except for lipofectamine groups to 35,000 cells/well in a 96 well plate. siRNA was mixed with counterion, left at RT for 3 days, then mixed with FBS containing media and given to cells. Live cell populations were selected after adding trypan blue to trypsinized cells using flow cytometry. Median fluorescein isothiocyanate (FITC) area, plotted as relative fluorescence units (R.F.U.) was utilized to compare uptake. FIG. 10D shows quantification of influence on cellular metabolism using 3-(4,5-dimethylthiazol-2-yl)-2,5-diphenyltetrazolium bromide (MTT) assay. 75 nM siRNA was given in the presence of FBS for all groups except for lipofectamine groups to 35,000 cells/well in a 96 well plate. siRNA was mixed with counterion, left at RT for 3 days, then mixed with FBS containing media and given to cells. Influence on cell viability after 48 hours was normalized to PBS treated cell viability.

FIG. 11 provides epifluorescence images of NIH3T3/GFP cells at 100× treated with 75 nM siRNA and of cells, in accordance with some embodiments of the present disclosure. HIP siRNA BA complexes were left at RT for 3 days before adding to cells. HIP and lipofectamine groups silence eGFP (note that lipofectamine reduces cell viability).

FIG. 12A provides quantification of pDNA HIP complex size with different counterions. HIP groups were composed of equal volumes of 1 mg/ml pDNA with 15 mg/ml of BA. pDNA was mixed with an equal volume of 1×PBS because counterions were dissolved in 1×PBS. All groups were characterized without prolonged incubation. Samples were measured in triplicates where a z-average hydrodynamic diameter distribution was calculated based on sample intensity. FIG. 12B provides quantification of luciferase encoded pDNA HIP delivery using a luciferase assay in 15,000 cells/well of HeLa cells in a 96 well plate 72 hours after treatment. 2 μg pDNA was given in the presence of FBS for all groups except for lipofectamine groups. pDNA was mixed with counterion, then mixed with FBS containing media and given to cells. FIG. 12C provides a demonstration of mammalian cell pDNA transfection using Expifectamine™ or HIP. Expifectamine requires an additional day of maintenance of adding an enhancer whereas HIP requires no enhancers. FIG. 12D provides HA stalk encoded pDNA Expi293F™ mammalian cell transfection using 50 μg of plasmid DNA for 5 days in Expi293F cells. The Expi293F™ cells transfected by using ExpiFectamine™ 293 Reagent or benethamine HIP were pelleted to harvest HA stalk and proteins were extracted from the cells using sonication in a buffer containing 20 mM imidazole, 300 mM NaCl, 50 mM NaH₂PO₄. Cell debris were removed by centrifuging, the supernatant was incubated with Ni-NTA agarose resin overnight, and the HA stalk was washed with 30 mL of 50 mM imidazole buffer followed by elution with 10 mL of 300 mM imidazole buffer. The buffer of purified proteins was exchanged with PBS and the purified HA stalk was analyzed by western blot. Prior to western blot, a mixture of 30 μL of purified HA stalk and 10 μL of Laemmeli buffer was heated at 95 for 5 min and loaded into a 12% SDS-PAGE gel. The SDS-PAGE electrophoresis was run for 70 min at 150 V in Tris-Glycine buffer.

FIG. 13 provides Lysosomal Associated Membrane Protein 1 (LAMP1) gene silencing measured using flow cytometry of cells from each organ (lung, liver, pancreas, and kidney) using a 1.5 mg/kg dose to BALB/c mice 48 hours after an intraperitoneal injection.

FIG. 14 provides Lysosomal Associated Membrane Protein 1 (LAMP1) gene silencing measured using flow cytometry of cells from the kidney labelled with endothelial (CD31+) or epithelial (E-cadherin+) antibodies using a 1.5 mg/kg dose to BALB/c mice 48 hours after an intraperitoneal injection.

### DETAILED DESCRIPTION

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

In vitro and ex vivo transfection of protein and nucleic acids requires carriers to achieve functional intracellular delivery for therapeutic, manufacturing, or research purposes. Existing transfection agents are characterized by high cost, low efficiency, and toxicity. As described herein, simple mixing of a hydrophobic counterion with a charged biomacromolecule cargo formed a complex capable of intracellular delivery without toxicity. Hydrophobic ion pairing resulted in functional cytosolic protein delivery, gene silencing comparable to lipofectamine RNAiMax without toxicity and millions of times cheaper, and plasmid transfection enabled mammalian protein expression over 6,000 times cheaper than ExpiFectamine™. Successful delivery depended on the ratio of cargo to hydrophobic ion. Altogether, this work reveals an inexpensive generalizable in vitro tool for cytosolic delivery of proteins and nucleic acids without toxicity.

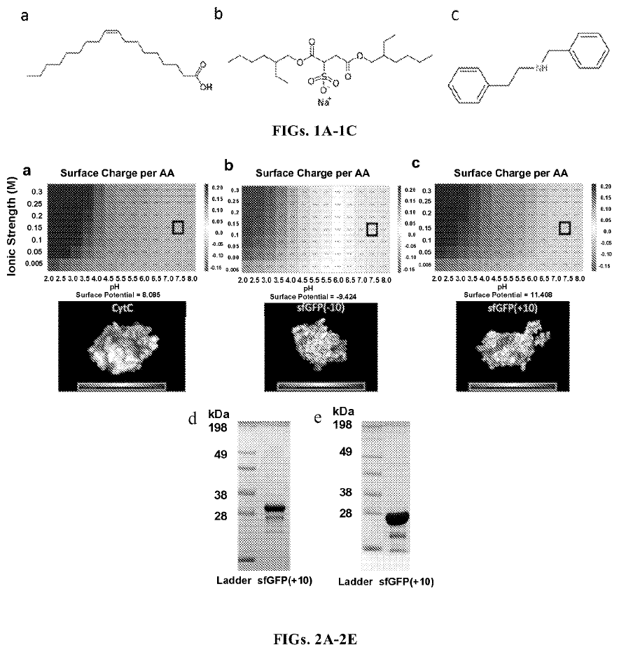

While HIP has been used to enable encapsulation of biomacromolecules in various types of nanocarriers, herein is described how HIP alone enabled cytosolic delivery of complexed protein and nucleic acids. This work evaluates HIP as simple, inexpensive, non-toxic in vitro transfection tool. The selected counterions in this work have one charge group either negative or positive and no head group making them unique from self-emulsifying drug delivery systems, polyelectrolyte complexes, and lipid nanoparticles (FIGS. 1A-1C). The ability of proteins and nucleic acids to pair with hydrophobic ions and be delivered functionally into the cytosol of various cell types without toxicity was assessed.

An exemplary embodiment of the present disclosure provides an intracellular delivery complex, comprising at least one biomacromolecule, each of the at least one biomacromolecules having a first ionic net charge, and at least one hydrophobic counterion, each of the at least one hydrophobic counterions having a second ionic net charge opposite the first ionic net charge, and wherein the complex is not encapsulated in a nanocarrier.

In some embodiments, the first ionic net charge has a first magnitude and the second ionic net charge has a second magnitude different than the first magnitude.

In some embodiments, the at least one biomacromolecules is a protein, peptide, or nucleic acid. For example, in some embodiments, the at least one biomacromolecules comprises a molecule of cytochrome C, a molecule of SiRNA, or a molecule of plasmid DNA. Other suitable biomacromolecules include antisense oligonucleotides, mRNA, micro RNA, aptamers, or any oligonucleotide.

The term “nucleic acid” refers to a polymer containing at least two nucleotides. “Nucleotides” contain a sugar deoxyribose (DNA) or ribose (RNA), a base, and a phosphate group. Nucleotides are linked together through the phosphate groups (although synthetic nucleic acids may be prepared using nucleotide linkers other than phosphate groups). “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. DNA may be in the form of antisense, plasmid DNA, parts of a plasmid DNA, pre-condensed DNA, product of a polymerase chain reaction (PCR), vectors (P1, PAC, BAC, YAC, artificial chromosomes), expression cassettes, chimeric sequences, chromosomal DNA, or derivatives of these groups. RNA may be in the form of siRNA, mRNA, microRNA, aptamers, or derivatives of these.

The term “hydrophobic counterion” refers to an ion with at least one hydrophobic domain, such as an alkyl tail or aromatic ring, that is capable of forming an ionic interaction with an oppositely charged hydrophilic molecule. The complexation of the hydrophobic counterion increases hydrophobicity by masking the charge of the hydrophilic molecule, as well as by coating the molecule's surface area with hydrophobic domains that exclude water (Ristroph and Prud′homme, “Hydrophobic Ion Pairing: Encapsulating Small Molecules, Peptides, and Proteins into Nanocarriers,” Nanoscale Advances, 1 (11): 4207-4237 (2019).

In some embodiments, the at least one hydrophobic counterion comprises one charge group and no hydrophilic head group. In some embodiments, the at least one hydrophobic counterion has more than one charge group. In some embodiments, the at least one hydrophobic counterions is a cation, while in other embodiments, the at least one hydrophobic counterions is an anion. In some embodiments the at least one hydrophobic counterions is selected from the group consisting of oleic acid, sodium docusate, benethamine, arginine hexadecanoyl ester, arginine-nonyl ester, lauramide arginine ethyl ester, 2-naphthalene sulfonic acid, carboxy methyl polyethylene glycol, cholesterol hemisuccinate, decanoic acid, docosahexaenoic acid, linoleic acid, sodium acetate, vitamin E succinate, dodecylamine, maprotiline, N,N-dimethyl dodecylamine, N,N-dimethyl hexylamine, N,N-dimethyl octadecylamine, stearylamine, tetrabutyl ammonium bromide, and triethylamine. In some embodiments the at least one biomacromolecule comprises a molecule of cytochrome C and the at least one hydrophobic counterion comprises an oleic acid ion. In other embodiments, the at least one biomacromolecule comprises a molecule of cytochrome C and the at least one hydrophobic counterion comprises a sodium docusate ion. In other embodiments, the at least one biomacromolecule comprises a molecule of siRNA and the at least one hydrophobic counterion comprises a benethamine ion. In other embodiments, the at least one biomacromolecule comprises a molecule of plasmid DNA and the at least one hydrophobic counterion comprises a benethamine ion.

In some embodiments, a molar ratio of the biomacromolecules to the hydrophobic counterions in the complex is about 1:10⁻¹⁰ to about 1:396. In some embodiments, the molar ratio is about 1:10⁻¹⁰ to about 1:10⁻⁹, about 1:10⁻¹⁰ to about 1:10⁻⁸, about 1:10⁻¹⁰ to about 1:10⁻⁷, about 1:10⁻¹⁰ to about 1:10⁻⁶, about 1:10⁻¹⁰ to about 1:10⁻⁵, about 1:10⁻¹⁰ to about 1:104, about 1:10⁻¹⁰ to about 1:10⁻³, about 1:10⁻¹⁰ to about 1:10⁻², about 1:10⁻¹⁰ to about 1:10⁻¹, about 1:10⁻¹⁰ to about 1:1, about 1:10⁻¹⁰ to about 1:10, about 1:10⁻¹⁰ to about 1:100, about 1:10⁻¹⁰ to about 1:200, about 1:10⁻¹⁰ to about 1:300, about 1:10⁻¹⁰ to about 1:400, about 1:10⁻⁸ to about 1:10⁻⁷, about 1:10⁻⁸ to about 1:10⁻⁶, about 1:10⁻⁸ to about 1:105, about 1:10⁻⁸ to about 1:10⁻⁴, about 1:10⁻⁸ to about 1:10⁻³, about 1:10⁻⁸ to about 1:10⁻², about 1:10⁻⁸ to about 1:10⁻¹, about 1:10⁻⁸ to about 1:1, about 1:10⁻⁸ to about 1:10, about 1:10⁻⁸ to about 1:100, about 1:10⁻⁸ to about 1:200, about 1:10⁻⁸ to about 1:300, about 1:10⁻⁸ to about 1:400, about 1:10⁻⁶ to about 1:10⁻⁵, about 1:10⁻⁶ to about 1:104, about 1:10⁻⁶ to about 1:10⁻³, about 1:10⁻⁶ to about 1:10⁻², about 1:10⁻⁶ to about 1:10⁻¹, about 1:10⁻⁶ to about 1:1, about 1:10⁻⁶ to about 1:10, about 1:10⁻⁶ to about 1:100, about 1:10⁻⁶ to about 1:200, about 1:10⁻⁶ to about 1:300, about 1:10⁻⁶ to about 1:400, about 1:10⁻⁴ to about 1:10⁻³, about 1:10⁻⁴ to about 1:10⁻², about 1:10⁻⁴ to about 1:10⁻¹, about 1:104 to about 1:1, about 1:10⁻⁴ to about 1:10, about 1:10⁻⁴ to about 1:100, about 1:10⁻⁴ to about 1:200, about 1:104 to about 1:300, about 1:10⁻⁴ to about 1:400, 1:10⁻² to about 1:10⁻¹, about 1:10⁻² to about 1:1, about 1:10⁻² to about 1:10, about 1:10⁻² to about 1:100, about 1:10⁻² to about 1:200, about 1:10⁻² to about 1:300, about 1:10⁻² to about 1:400, about 1:1 to about 1:10, about 1:1 to about 1:50, about 1:1 to about 1:100, about 1:1 to about 1:200, about 1:10 to about 1:300, about 1:1 to about 1:396, about 1:1 to about 1:400, about 1:40 to about 1:50, about 1:40 to about 1:100, about 1:40 to about 1:200, about 1:40 to about 1:300, about 1:40 to about 1:396, about 1:40 to about 1:400, about 1:60 to about 1:100, about 1:60 to about 1:200, about 1:60 to about 1:300, about 1:60 to about 1:396, about 1:60 to about 1:400, about 1:80 to about 1:100, about 1:80 to about 1:200, about 1:80 to about 1:300, about 1:80 to about 1:396, about 1:80 to about 1:400, about 1:100 to about 1:200, about 1:100 to about 1:300, about 1:100 to about 1:396, about 1:100 to about 1:400, about 1:200 to about 1:300, about 1:200 to about 1:396, about 1:200 to about 1:400, about 1:300 to about 1:396, or about 1:300 to about 1:400.

In some embodiments, the at least one biomacromolecule and the at least one hydrophobic counterion are present in a charge ratio of about 1:10⁻¹¹ to about 1:44. In some embodiments the at least one biomacromolecule and the at least one hydrophobic counterion are present in a charge ratio of about 1:10⁻¹¹ to about 1:10⁻¹⁰, about 1:10⁻¹¹ to about 1:10⁻⁹, about 1:10⁻¹¹ to about 1:10⁻⁸, about 1:10⁻¹¹ to about 1:10⁻⁹, about 1:10⁻¹¹ to about 1:10⁻⁸, about 1:10⁻¹¹ to about 1:10⁻⁷, about 1:10⁻¹¹ to about 1:10⁻⁶, about 1:10⁻¹¹ to about 1:105, about 1:10⁻¹¹ to about 1:104, about 1:10⁻¹¹ to about 1:10⁻³, about 1:10⁻¹¹ to about 1:10⁻², about 1:10⁻¹¹ to about 1:10⁻¹, about 1:10⁻¹¹ to about 1:1, about 1:10⁻¹¹ to about 1:10, about 1:10⁻¹¹ to about 1:20, about 1:10⁻¹¹ to about 1:30, about 1:10⁻¹¹ to about 1:40, 1:10⁻¹¹ to about 1:50, about 1:10⁻⁹ to about 1:10⁻⁸, about 1:10⁻⁹ to about 1:10⁻⁷, about 1:10⁻⁹ to about 1:10⁻⁶, about 1:10⁻⁹ to about 1:105, about 1:10⁻⁹ to about 1:10⁻⁴, about 1:10⁻⁹ to about 1:10⁻³, about 1:10⁻⁹ to about 1:10⁻², about 1:10⁻⁹ to about 1:10⁻¹, about 1:10⁻⁹ to about 1:1, about 1:10⁻⁹ to about 1:10, about 1:10⁻⁹ to about 1:20, about 1:10⁻⁹ to about 1:30, about 1:10⁻⁹ to about 1:40, 1:10⁻⁹ to about 1:50, about 1:10⁻⁷ to about 1:10⁻⁶, about 1:10⁻⁷ to about 1:10⁻⁵, about 1:10⁻⁷ to about 1:104, about 1:10⁻⁷ to about 1:10⁻³, about 1:10⁻⁷ to about 1:10⁻², about 1:10⁻⁷ to about 1:10⁻¹, about 1:10⁻⁷ to about 1:1, about 1:10⁻⁷ to about 1:10, about 1:10⁻⁷ to about 1:20, about 1:10⁻⁷ to about 1:30, about 1:10⁻⁷ to about 1:40, 1:10⁻⁷ to about 1:50, about 1:10⁻⁵ to about 1:104, about 1:10⁻⁵ to about 1:10⁻³, about 1:10⁻⁵ to about 1:10⁻², about 1:10⁻⁵ to about 1:10⁻¹, about 1:105 to about 1:1, about 1:105 to about 1:10, about 1:105 to about 1:20, about 1:10⁻⁵ to about 1:30, about 1:10⁻⁵ to about 1:40, 1:10⁻⁵ to about 1:50, about 1:10⁻³ to about 1:10⁻², about 1:10⁻³ to about 1:10⁻¹, about 1:103 to about 1:1, about 1:10⁻³ to about 1:10, about 1:10⁻³ to about 1:20, about 1:103 to about 1:30, about 1:10⁻³ to about 1:40, 1:103 to about 1:50, about 1:10⁻² to about 1:10⁻¹, about 1:10⁻² to about 1:1, about 1:10⁻² to about 1:10, about 1:10⁻² to about 1:20, about 1:10⁻² to about 1:30, about 1:10⁻² to about 1:40, 1:10⁻² to about 1:50, about 1:10⁻¹ to about 1:1, about 1:10⁻¹ to about 1:10, about 1:10⁻¹ to about 1:20, about 1:10⁻¹ to about 1:30, about 1:10⁻¹ to about 1:40, about 1:10⁻¹ to about 1:50, about 1:1 to about 1:10, about 1:1 to about 1:20, about 1:1 to about 1:30, about 1:1 to about 1:40, or about 1:1 to about 1:50.

In some embodiments, the at least one biomacromolecule and the at least one hydrophobic counterion are present in a mass ratio of about 1:10⁻¹² to about 1:15. In some embodiments the at least one biomacromolecule and the at least one hydrophobic counterion are present in a mass ratio of about 1:10⁻¹² to about 1:10⁻¹¹, about 1:10⁻¹² to about 1:10⁻¹⁰, about 1:10⁻¹² to about 1:10⁻⁹, about 1:10⁻¹² to about 1:10⁻⁸, about 1:10⁻¹² to about 1:10⁻⁷, about 1:10⁻¹² to about 1:10⁻⁶, about 1:10⁻¹² to about 1:10⁻⁵, about 1:10⁻¹² to about 1:104, about 1:10⁻¹² to about 1:10⁻³, about 1:10⁻¹² to about 1:10⁻², about 1:10⁻¹² to about 1:10⁻¹, about 1:10⁻¹² to about 1:1, about 1:10⁻¹² to about 1:5, about 1:10⁻¹² to about 1:10, about 1:10⁻¹² to about 1:15, about 1:10⁻¹² to about 1:20, about 1:10⁻¹⁰ to about 1:10⁻⁹, about 1:10⁻¹⁰ to about 1:10⁻⁸, about 1:10⁻¹⁰ to about 1:10⁻⁷, about 1:10⁻¹⁰ to about 1:10⁻⁶, about 1:10⁻¹⁰ to about 1:10⁻⁵, about 1:10⁻¹⁰ to about 1:10⁻⁴, about 1:10⁻¹⁰ to about 1:10⁻³, about 1:10⁻¹⁰ to about 1:10⁻², about 1:10⁻¹⁰ to about 1:10⁻¹, about 1:10⁻¹⁰ to about 1:1, about 1:10⁻¹⁰ to about 1:5, about 1:10⁻¹⁰ to about 1:10, about 1:10⁻¹⁰ to about 1:15, about 1:10⁻¹⁰ to about 1:20, about 1:10⁻⁸ to about 1:10⁻⁷, about 1:10⁻⁸ to about 1:10⁻⁶, about 1:10⁻⁸ to about 1:10⁻⁵, about 1:10⁻⁸ to about 1:104, about 1:10⁻⁸ to about 1:10⁻³, about 1:10⁻⁸ to about 1:10⁻², about 1:10⁻⁸ to about 1:10⁻¹, about 1:10⁻⁸ to about 1:1, about 1:10⁻⁸ to about 1:5, about 1:10⁻⁸ to about 1:10, about 1:10⁻⁸ to about 1:15, about 1:10⁻⁸ to about 1:20, about 1:10⁻⁶ to about 1:10⁻⁵, about 1:10⁻⁶ to about 1:104, about 1:10⁻⁶ to about 1:10⁻³, about 1:10⁻⁶ to about 1:10⁻², about 1:10⁻⁶ to about 1:10⁻¹, about 1:10⁻⁶ to about 1:1, about 1:10⁻⁶ to about 1:5, about 1:10⁻⁶ to about 1:10, about 1:10⁻⁶ to about 1:15, about 1:10⁻⁶ to about 1:20, about 1:10⁻⁴ to about 1:10⁻³, about 1:10⁻⁴ to about 1:10⁻², about 1:10⁻⁴ to about 1:10⁻¹, about 1:10⁻⁴ to about 1:1, about 1:10⁻⁴ to about 1:5, about 1:10⁻⁴ to about 1:10, about 1:10⁻⁴ to about 1:15, about 1:10⁻⁴ to about 1:20, about 1:10⁻² to about 1:10⁻¹, about 1:10⁻² to about 1:1, about 1:10⁻² to about 1:5, about 1:10⁻² to about 1:10, about 1:10⁻² to about 1:15, about 1:10⁻² to about 1:20, about 1:1 to about 1:5, about 1:1 to about 1:10, about 1:1 to about 1:15, about 1:1 to about 1:20, about 1:5 to about 1:10, about 1:5 to about 1:15, about 1:5 to about 1:20, about 1:10 to about 1:15, or about 1:10 to about 1:20.

Another embodiment of the present disclosure provides a method of delivering a biomacromolecule into a cell. This method comprises providing at least one biomacromolecule, each of the at least one biomacromolecules having a first ionic net charge, providing at least one hydrophobic counterion, each of the at least one hydrophobic counterions having a second ionic net charge opposite the first ionic net charge, mixing the at least one biomacromolecule with the at least one hydrophobic counterion under conditions effective to form a complex capable of entering a cell, wherein the complex is not encapsulated in a nanocarrier.

Suitable biomacromolecules and hydrophobic counterions are those of the present disclosure as described herein.

In some embodiments, the complex is capable of entering a cell via endocytosis. In some embodiments, the method can further comprise administering the complex to a subject such that the complex enters the cell via endocytosis.

As described herein a subject should be broadly interpreted to include, but is not limited to, a living organism, a cell, a tissue, an organ, or other biological component of a biological specimen. Said administering the complex to a subject can include administering the complex to any biological specimen using in vitro, ex vivo, and/or in vivo techniques.

In some embodiments of the method, the mixing is carried out in the presence of a solvent. In some embodiments, the solvent is water. In some embodiments, the solvent is an aqueous buffer.

Another embodiment of the present disclosure provides a method of administering a biomacromolecule into a cell. This method comprises providing an intracellular delivery complex, comprising at least one biomacromolecule, each of the at least one biomacromolecules having a first ionic net charge; and providing at least one hydrophobic counterion, each of the at least one hydrophobic counterions having a second ionic net charge opposite the first ionic net charge, and wherein the complex is not encapsulated in a nanocarrier; and administering the intracellular delivery complex to a subject, such that the complex enters a cell of the subject.

Suitable biomacromolecules and hydrophobic counterions are those of the present disclosure as described herein.

As used herein, “not encapsulated in a nanocarrier” should not be interpreted to mean that the intracellular delivery complex cannot be administered to a subject via capsule or other mechanism for oral administration.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

### EXAMPLES

The following Examples are presented to illustrate various aspects of the present application, but are not intended to limit the scope of the claimed application.

### Example 1—Materials and Methods for Examples 2-6

### Materials

Benethamine (N-benzyl-2-phenylthanamine) (catalog number: APOH316Cde11), OA (catalog number: 007501), SD (catalog number: D4422), CytC (catalog number: 63103), fetal bovine serum (FBS), trypsin-EDTA, and imidazole were obtained from Sigma-Aldrich. Butanol was obtained from Millipore Sigma. Liptofectamine RNAiMAX Transfection Reagent (catalog number: 13778030) and siRNA silencing eGFP (catalog number: AM4626) were from Fisher Scientific. NIH 3T3 fibroblasts (GFP3T3) reporter cells (catalog number: AKR-214) was from CellBio Labs. Dulbecco's modified Eagle's medium (DMEM) was from Thermofisher.

### Bacterial Protein Expression & Purification

The pET28a-sfGFP-His plasmids with sfGFP variants were purchased from Genscript. sfGFP variants were expressed in Escherichia coli strain BL21 (DE3) star. To express these proteins, 1 L of lysogeny broth (LB) containing 50 mg kanamycin was inoculated with 5 mL of overnight culture at 37° C. then induced with 1 mM isopropyl β-D-1 thiogalactopyranoside (IPTG) when the optical density was greater than 0.7. After 5 hours of expression, cultures were collected by centrifugation at 4000 g for 10 minutes. The pellets were resuspended in lysis buffer containing 300 mM NaCl, 50 mM NaH₂PO₄, and 10 mM imidazole and lysed by sonication. Next, the supernatant was collected after centrifugation at 10,000 rotations per minute (rpm) for 10 minutes and incubated with Ni-nitrilotriacetic acid (NTA) agarose resin (Qiagen) for at least 1 hour at 4° C. This mixture was applied to an Econo-Column (Biorad) and washed with 100 mL of 25 mM imidazole and 10 mL of elution was collected using 250 mM imidazole. Purity was verified using sodium dodecyl sulfate polyacrylamide gel electrophoresis (SDS-PAGE), then the proteins were buffer exchanged into phosphate buffered saline (PBS) by dialysis with three buffer exchanges at 4° C.

### Hydrophobic Ion Paired Complex Formation and Characterization

HIP complexes were formed by a simple mixing method. Cargo at a concentration of 1 mg/ml for proteins, 50 μM for siRNA, or 50 μg for pDNA were mixed with varying concentrations of SD, OA, or BA at equal volumes. If incubating for longer than immediate use, the complexes were left at room temperature and protected from light because many counterions are light sensitive. Log D experiments were performed by mixing 0.5 ml of water with 0.5 mL butanol then adding 100 μL cargo and HIP complexes with 0.5 mg/mL cargo. After mixing at 150 rpm overnight to ensure phase separation, concentration in each phase was measured using A260/280 NanoDrop values to calculate the partition coefficient.

Size and zeta potential were measured using dynamic light scattering (DLS) using Malvern Instruments Zetasizer NanoZS intensity mode with a 4 mW He—Ne laser with 633 nm wavelength to detect backscattering) (173° using 1× for DLS or 0.1× PBS for zeta potential at pH 7.4 solvent conditions and protein material selection. Z-average values were used for HIP complex size. Electrophoretic mobility was converted to zeta potential using the Smoluchowski approximation. Imaging of solutions was performed using an epifluorescence microscope (Zeiss Axio Observer Z1) using a 100× oil immersion lens. Transmission electron microscopy (TEM) grids were prepared using 10 μL sample on copper grids, letting the sample adhere for 5 minutes, washing in water for 30 seconds, staining with 1% phosphotungstic acid for 10 seconds, washing again for 30 seconds, and drying overnight. A JEOL 100 CX-II TEM was utilized to visualize samples.

### Cell Culture

HeLa cells (ATCC) were seeded at 15,000 cells/well using Dulbecco's modified Eagle medium (DMEM) supplemented with 10% fetal bovine serum (FBS) in 96 well plates for 24 hours prior to treatment, washed with PBS, then treated with HIP complexes at 37° C. in a humidified environment containing 5% CO₂ for 24 or 48 hours. NIH 3T3 fibroblast cells expressing eGFP (Cell BioLabs) were seeded at 35,000 cells/well in a 96 well plate for 24 hours before treatment using DMEM supplemented with 10% FBS. All groups besides lipofectamine were treated with DMEM and 10% FBS for a final well concentration of 75 nM siRNA. After incubation for 24 hours, the media was changed to give all groups 10% FBS and cultured for another 24 hours at 37° C. in a humidified environment containing 5% CO₂. NIH 3T3 fibroblast cells were cultured with the same conditions as HeLa cells.

K-562 (human erythromyeloblastoid leukemia cell line ATCC, UK; CCL-243), originally derived from a patient with blast crisis of chronic myeloid leukemia was chosen to model acute myeloid leukemia and were seeded at 25,000 cells/well in 96 well plates for 24 hours prior to treatment, washed with PBS, then treated with HIP complexes at 37° C. in a humidified environment containing 5% CO₂ for 48 hours. Cells were cultivated in Roswell Park Memorial Institute 1640 Medium (RPMI; Corning, Manassas, VA) supplemented with 10% v/v fetal bovine serum (FBS, heat inactivated; Gibco, NY).

### Protein Expression by Transfecting Mammalian Cells and Western Blot Analysis

For suspension culture, Expi293F™ cells were maintained in 50 mL of Expi293 Expression Medium (Thermo Fisher Scientific) in 250 mL polycarbonated vented Erlenmeyer flasks (Thermo Fisher Scientific) at 37, 8% CO₂ on a shaker at 125 rpm. After the cell density reached 3×10⁶ cells/mL with >95% viability, Expi293F™ cells were transfected in 50 mL of fresh medium using Expi293™ Expression System kit (Thermo Fisher Scientific) as per manufacturer's instructions. Briefly, 160 μL of ExpiFectamine™ 293 Reagent was mixed with 2.8 mL Opti-MEM I Reduced Serum Medium and incubated at room temperature for 5 min. Then, the diluted ExpiFectamine™ 293 Reagent was added to 50 μg of pcDNA3.1 plasmid encoding HA stalk diluted in 3 mL Opti-MEM I Reduced Serum Medium. The mixture was incubated at room temperature for 20 min to allow the plasmid DNA complexation. The DNA complexes were then added to the 50 mL of 3×10⁶ cells/mL Expi293F™ cells in suspension, and the cells were culture at 37, 8% CO₂ on a shaker at 125 rpm. After 20 hours of the incubation, the transfected Expi293F™ cells were incubated with 300 μL of Transfection Enhancer 1 and 3 mL of Transfection Enhancer 2 for 4 days. For HIP-mediated transient transfection, 100 μL of 15 mg/mL benethamine HIP was mixed with 100 μL of 500 ng/μL pcDNA3.1 plasmid encoding HA stalk and incubated for 20 min, and the mixture was slowly added to the 50 mL of 3×10⁶ cells/mL Expi293F™ cells in suspension. The transfected Expi293F™ cells were cultured for 5 days.

The Expi293F™ cells transfected by using ExpiFectamine™ 293 Reagent or benethamine HIP were pelleted 5 days post-transfection via centrifugation to harvest HA stalk. The proteins were extracted from the cells by sonicating the cell pellets in 40 mL of a lysis buffer containing 20 mM imidazole, 300 mM NaCl, 50 mM NaH₂PO₄. After the cell lysis, the cell debris were removed by centrifuging at 10,000 rcf for 25 min at 4C. The supernatant was incubated with Ni-NTA agarose resin overnight, and the HA stalk was washed with 30 mL of 50 mM imidazole buffer followed by elution with 10 mL of 300 mM imidazole buffer. The buffer of purified proteins was exchanged with PBS using 3 kDa molecular weight cut-off Amicon Ultra-4 Centrifugal Filter Unit (Millipore Sigma). The purified HA stalk was analyzed by western blot. Prior to western blot, a mixture of 30 μL of purified HA stalk and 10 μL of Laemmeli buffer was heated at 95 for 5 min and loaded into the stacking layer of a 12% SDS-PAGE gel. The SDS-PAGE electrophoresis was run for 70 min at 150 V in Tris-Glycine buffer. Then, the proteins in the gel were transferred to the membrane by performing western blot as per Bio-RAD Mini-Protean western blotting protocol. The membrane was blocked with a blocking buffer consisting of 5% w/v dry milk, 1×PBS, and 0.1% Tween-20 and incubated with Penta-His Alexa Fluor 488 Conjugate (Qiagen) to detect His tagged HA stalk from the membrane.

Cellular Uptake & GFP Knockdown

Before flow cytometry analysis, cells were detached using trypsin and resuspended in PBS with trypan blue to quench background signal. The sfGFP fluorescence of cells was measured using FITC on the CytoFLEX flow cytometer with stopping conditions of 420 seconds and 10,000 cells (BD Bioscience). Gating was done on PBS groups to isolate single cells and compare increase or decrease in GFP signal.

### Cell Viability Assay

The in vitro toxicity was quantified using a methylthiazoltetrazolium (MTT) assay. After treatment, the media was removed, cells were washed with PBS, then replaced with fresh media with 10 μL of 5 mg/ml MTT solution (Biotium). The cells were incubated for 4 hours at 37° C. in a humidified environment containing 5% CO₂. Then, 200 μL of dimethyl sulfoxide (DMSO) was added to the mixture to formazan crystals. Absorbances of solutions were measured to remove background and obtain viability (570 nm and 630 nm). Cell viability was calculated by using the number of viable cells in a treated group relative to the PBS control group. Similarly, an MTS assay was performed to determine cell viability in our organoid model which does not require re-solubilization with DMSO and uses an absorbance of 490 nm.

### Hemolytic Assay & Epifluorescent Imaging

Washed red blood cells (RBC's) were diluted to a 5% v/v concentration in PBS. 100 μL of nanoparticles or treatment were incubated with 10 μL RBC's at 37° C. for 1 hour in a 96 well plate. After incubation with HIP treatment groups, the microplate reader was used to measure released hemoglobin from membrane disruption using 541 nm and subtracting background at 561 nm, then normalizing hemolytic activity to 5% Triton X-100. For imaging RBC's and NIH3T3/eGFP cells, an epifluorescent microscope (Zeiss Axio Observer Z1) with an 40× lens.

### Statistics

One-way ANOVA was used to compare 3 or more groups by using the F test for statistical significance by comparing multiple means by calculating error between all comparisons. When less than 3 groups were compared, a T-test was used for an individual two-way comparison.

### Example 2—Development of Intracellular Protein Delivery Using HIP

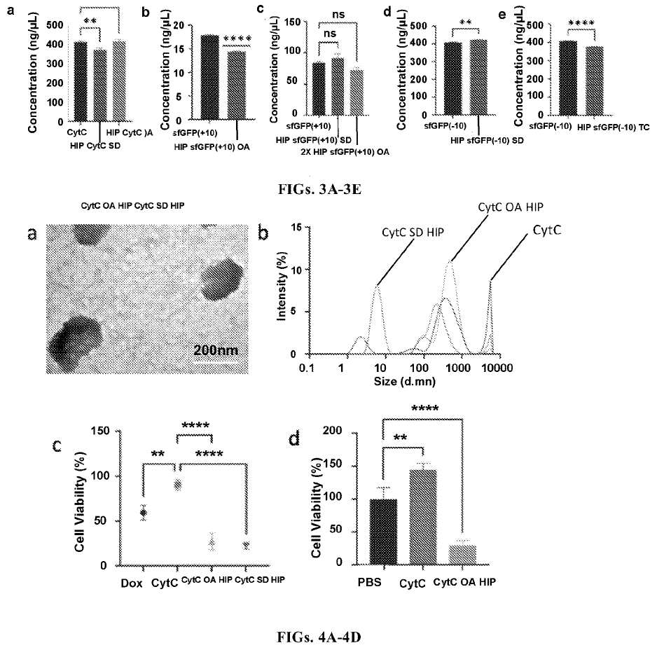

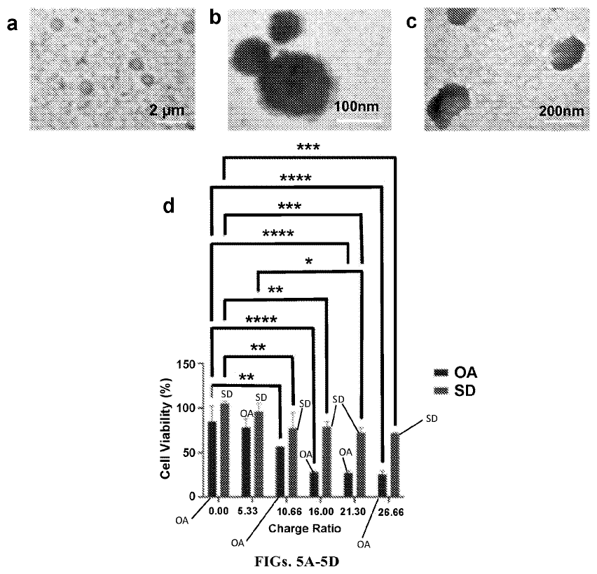

To show that HIP complexes deliver functional protein to the cytosol, CytC was delivered as its target is the mitochondrial membrane. CytC is a 12.4 kDa protein with a surface potential of +8.08 at pH 7.4 (FIG. 2) (Mirdita et al., “ColabFold: Making Protein Folding Accessible to All,” Nature Methods, 19 (6): 679-682 (2022); Hebditch and Warwicker, “Web-Based Display of Protein Surface and PH-Dependent Properties for Assessing the Developability of Biotherapeutics,” Scientific Reports, 9 (1): 1969 (2019)). To make HIP complexes, counterions were suspended into PBS, then mixed 2-3 times with a pipette with an equal volume of CytC and used without prolonged incubation in ratios resulting in less than 30% decrease in protein fluorescence (Table 1, FIG. 3). When a charge ratio of 1 CytC: 16 OA is mixed, the HIP complexes form oblong dense clusters (FIG. 4A). At lower mass ratios, the HIP CytC OA complexes are less oblong and more spherical structures (FIG. 5). CytC was paired with OA in a 1:16 mass ratio and with SD in a 1:13 mass ratio to form CytC HIP complexes where both complexes were 203±130 and 317±9.7 nm in diameter while CytC was 729±460 nm in diameter (FIG. 4B). CytC had a polydispersity index (PDI) of 0.542±0.17 while HIP CytC SD had a PDI of 0.274±0.108 and HIP CytC OA had a PDI of 0.480±0.07. Both HIP CytC OA and HIP CytC SD demonstrated cytosolic delivery when treated in the presence of serum containing media resulting in high levels of HeLa apoptosis at a dose of 20 μM after 48 hours (FIGS. 4A and 4C). Additionally, to examine the role of concentration of counterion on cytosolic delivery, varying ratios of CytC to OA or SD were complexed with CytC and incubated with HeLa cells where complex size changed with changing charge ratio of OA (FIG. 5). Using a charge ratio of 1:10.66 or higher resulted in functional CytC delivery (FIG. 5). However, there was a critical ratio (1:11) needed for functional intracellular delivery for both counterions, at least within 48 hours.

TABLE 1
HIP Protein Complex Mass, Molar, and Charge Ratios
Charge ratios were calculated based on charged surface
accessible amino acid residues in cargo for proteins or
based on number of base pairs for nucleic acids.

| Cargo:Counterion | Mass Ratio | Molar Ratio | Charge Ratio |
|---|---|---|---|
| sfGFP(+10):OA | 1:4 | 1:396 | 1:35 |
| sfGFP(+10):SD | 1:1 | 1:62 | 1:5.4 |
| sfGFP(−10):BA | 1:1 | 1:132 | 1:14 |
| CytC:OA | 1:3 | 1:127 | 1:16 |
| CytC:SD | 1:4 | 1:108 | 1:13 |
| SIRNA:BA | 1:4 | 1:0.585 | 1:0.03 |
| pDNA:BA | 1:15 | 1:0.008 | 1:1.6 × 10⁻⁶ |

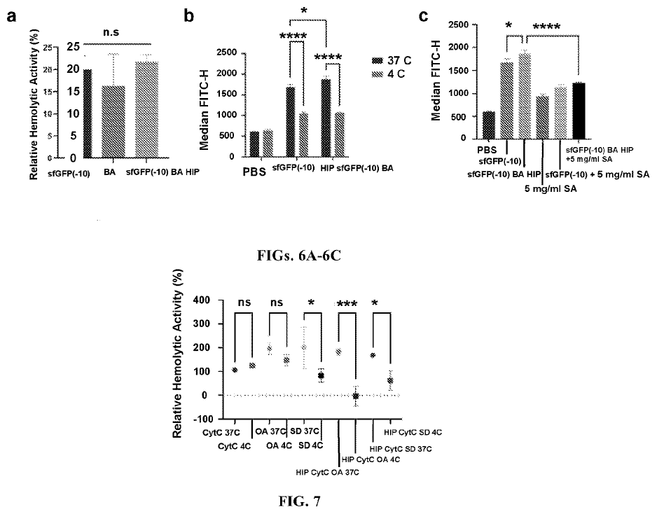

Cytosolic delivery suggests that complexes have some interaction with the endosomal membrane. This possibility was investigated using a hemolytic activity assay was performed to measure hemoglobin release from red blood cells at 37° C. and 4° C., to increase membrane stiffness from changes in lipid packing (Herrera et al., “Bioengineering Bacterially Derived Immunomodulants: A Therapeutic Approach to Inflammatory Bowel Disease,” ACS Nano, 11 (10): 9650-9662 (2017); Amaro et al., “Laurdan and Di-4-ANEPPDHQ Probe Different Properties of the Membrane,” Journal of Physics D: Applied Physics, 50 (13): 134004 (2017)). At 37° C., both anionic counterions, CytC, and HIP complexes disrupted the red blood cell (RBC) membrane resulting in greater than 100% relative hemolytic activity at the same concentration used in HIP CytC complexes (FIG. 6). SD's mechanism of action when used as a laxative is decreasing surface tension to increase water penetration into the stool (McRorieet et al., “Psyllium Is Superior to Docusate Sodium for Treatment of Chronic Constipation,” Alimentary Pharmacology & Therapeutics, 12 (5): 491-497 (1998)). CytC also acts as a membrane disruptor because CytC binds cardiolipin, a mitochondrial membrane glycerophospholipid (Eleftheriadis et al., “Cytochrome c as a Potentially Clinical Useful Marker of Mitochondrial and Cellular Damage,” Frontiers in Immunology, 7:279 (2016); Thong and Tsoukanova, “Cytochrome-c-Assisted Escape of Cardiolipin from a Model Mitochondrial Membrane,” Biochimica et Biophysica Acta (BBA)—Biomembranes, 1860 (2): 475-480 (2018)). When the temperature was reduced to 4° C., where the membrane is stiffer (Amaro et al., “Laurdan and Di-4-ANEPPDHQ Probe Different Properties of the Membrane. Journal of Physics D: Applied Physics, 50 (13): 134004 (2017)), all counterion groups had no difference in activity, except for SD which still remained high, while all HIP complexes had significantly lower hemolytic activity (FIG. 7).

### Example 3—HIP Complexes with Positive and Negatively Charged sfGFP Lack Mechanisms Influencing Cell Viability in HeLa Cells

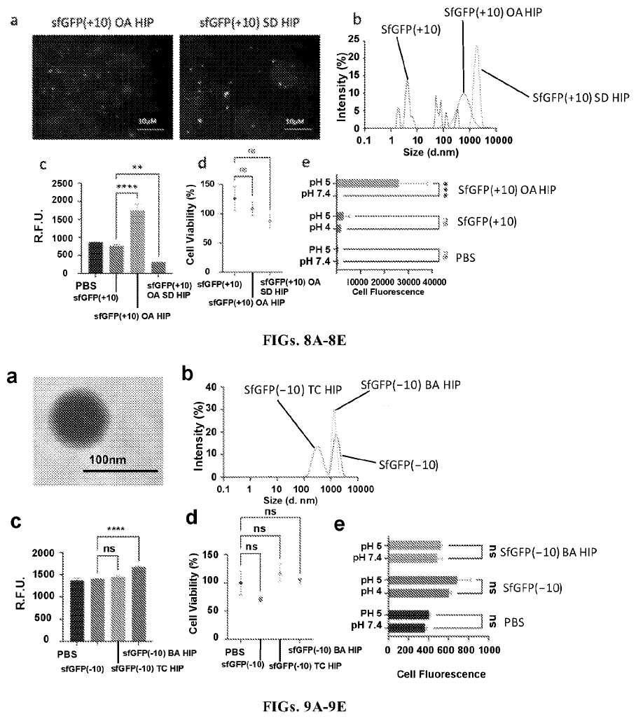

sfGFP(+10) was expressed in bacteria then purified using nickel affinity where purity was determined using an SDS-page gel (FIG. 2). To make HIP complexes, counterions were resuspended into 1× PBS, then mixed 2-3 times with a pipette with an equal volume of biomacromolecule and used without prolonged incubation at conditions which resulting in less than 30% decrease in protein fluorescence (Table 1, FIG. 3). The HIP complexes with a charge ratio of 1 sfGFP(+10): 35 OA complexes were 439±12 nm in diameter (<200 while the HIP complexes with a charge ratio of 1 sfGFP(+10): 5.4 SD complexes were 1730±229 nm in diameter, sfGFP(+10) alone was 4757±696 nm in diameter (FIGS. 8A and 8B). Protein had a PDI of 1.00±0.0, sfGFP(+10) OA HIP had a PDI of 0.369±0.06, and sfGFP(+10) SD HIP had a PDI of 0.144±0.06 indicating polydispersity reduced when counterion was complexed. Without counterion, sfGFP(+10) had a zeta potential of −3.4±0.9 mV and when complexed with OA at a charge ratio of 1:35 had a zeta potential of −57.4±2.2 mV. sfGFP(+10) OA HIP had significantly higher uptake than free protein and sfGFP(+10) SD HIP in HeLa cells at a 1 UM dose after 24 hours in the presence of serum containing media (FIG. 8C). Using SD resulted in lower uptake than free protein which could be caused by the significantly lower concentration of SD used. At this low charge ratio of SD, there is no increase in permeation ability for sfGFP(+10) complexes. When CytC OA complexes were given to HeLa cells in a charge ratio less than 1 CytC: 11 OA, no significant reduction in cell viability occurred pointing to a critical charge ratio (FIG. 5). To examine the toxicity of counterions, sfGFP(+10) HIP complexes were delivered to HeLa cells and their influence on cell metabolism was determined to be insignificant for charge ratios of 1 sfGFP(+10): 35 OA and 1 sfGFP(+10): 5.4 SD proving that the counterions complexed to non-toxic biomacromolecules do not negatively impact cell health at the conditions used for enhanced uptake (FIG. 8D). To examine membrane interactions of sfGFP(+10) OA HIP complexes, we utilized RBC as endosomal membrane models and measured extracellular binding of complexes (FIG. 8E). At acidic pH, sfGFP(+10) OA HIP complexes have significantly more binding and membrane interactions which led to improved delivery.

sfGFP(−10) was expressed in bacteria then purified using nickel affinity where purity was determined using an SDS-page gel (FIG. 2). To make HIP complexes, counterions were either diluted in 1×PBS if in aqueous form or resuspended into 1× PBS if in solid form, then mixed 2-3 times with a pipette with an equal volume of biomacromolecule and used without prolonged incubation using ratios resulting in less than 30% decrease in protein fluorescence (Table 1, FIG. 3). HIP sfGFP(−10) complexes were characterized to determine particle size, dispersity, and morphology. These complexes were core shell spheres (FIG. 9A). The charge of the complexes was influenced by the charge of BA, which resulted in an increase from −23.5±2.6 mV for sfGFP(−10) to −15.7±0.8 mV for sfGFP(−10) BA HIP complexes. To compare BA to another cationic counterion with multiple charge groups, N,N,N-trimethyl chitosan (TC) was selected. sfGFP(−10) was 436±37 nm in diameter, a charge ratio of 1 sfGFP(−10): 14 BA HIP resulted in 2010±161 nm in diameter, and a charge ratio of 1 sfGFP(−10): 5-20 TC HIP resulted in 289±3.7 nm in diameter. Polydispersities were 0.463±0.08, 0.620=0.10, and 0.171±0.02 demonstrating that TC produced the most monodisperse complexes. The sfGFP(−10) BA HIP complex resulted in improved uptake by HeLa cells using a charge ratio of 1:14, however not for TC (FIG. 9C). BA is not a known permeation enhancer, however is known to have selective antibacterial activity to gram negative bacteria at micromolar concentrations (Baidin et al., “Simple Secondary Amines Inhibit Growth of Gram-Negative Bacteria through Highly Selective Binding to Phenylalanyl-TRNA Synthetase,” J. Am. Chem. Soc., 143 (2): 623-627 (2021)). As a part of penicillin, which selectively disrupts bacterial cell membranes and not red blood cell membranes, BA plays an important role in membrane disruption (Yocum et al., “The Mechanism of Action of Penicillin. Penicillin Acylates the Active Site of Bacillus Stearothermophilus D-Alanine Carboxypeptidase,” Journal of Biological Chemistry, 255 (9): 3977-3986 (1980); Suwalsky et al., “Interaction of Penicillin G with the Human Erythrocyte Membrane and Models,” Zeitschrift für Naturforschung C, 51 (3-4): 243-248 (1996)). Additionally, sfGFP(−10) BA HIP and sfGFP(−10) TC HIP complexes had no negative impact on cell metabolism (FIG. 9D). Because TC did not improve uptake, further work includes only BA was a cationic counterion. Additionally, HIP complex uptake significantly decreases when endocytosis is inhibited (FIG. 6). To analyze endosomal membrane interactions, sfGFP(−10) BA HIP complex extracellular RBC binding was utilized (FIG. 9E). There were no significant differences at different pHs for sfGFP(−10) groups while sfGFP(+10) groups did pointing to how a change in pH influences sfGFP(+10) OA HIP complex ionic interactions while hydrophobic interactions are preserved. Lipid-membrane interactions are used in lipid nanoparticle delivery systems to escape the endosome and release cargos into the cytosol. Branched tail lipids are known to act as wedges within membrane bilayers to promote delivery (Hajj et al., “Branched-Tail Lipid Nanoparticles Potently Deliver MRNA In Vivo Due to Enhanced Ionization at Endosomal PH,” Small, 15 (6): 1805097 (2019)). Unlike lipid nanoparticle systems, the counterions in this work have no hydrophilic head group and contain one charged group, however, also rely on membrane interactions and complex hydrophobicity for intracellular delivery.

### Example 4—Effective siRNA Delivery in NIH3T3/eGFP Cells Significantly Cheaper than Lipofectamine

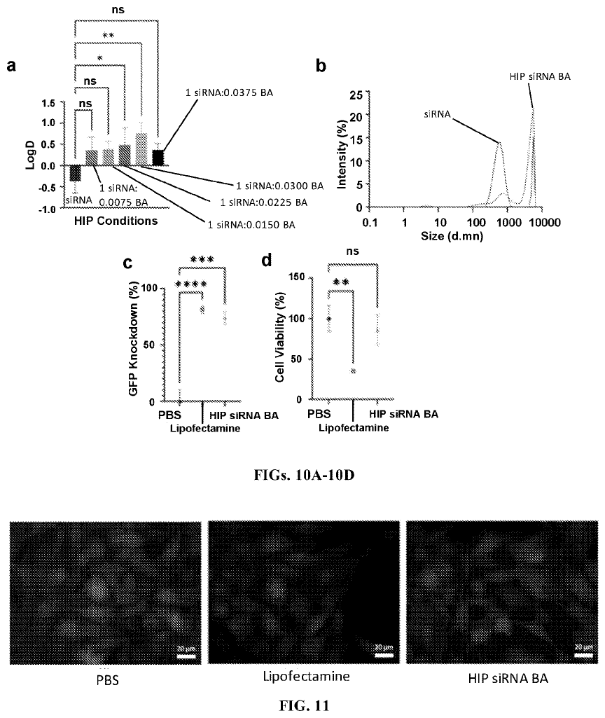

To make HIP complexes, counterions were resuspended into 1× PBS, then mixed 2-3 times with a pipette with an equal volume of biomacromolecule (Table 1). Because siRNA is much smaller than the proteins used in this work, a Log D partition coefficient was utilized to measure change in hydrophobicity. Log D values could not be determined for proteins in this work due to butanol induced unfolding. The Log D partition coefficient indicates more hydrophobic compound with positive values and more hydrophilic compounds with negative values (Wibel et al., “Hydrophobic Ion Pairing (HIP) of (Poly) Peptide Drugs: Benefits and Drawbacks of Different Preparation Methods,” European Journal of Pharmaceutics and Biopharmaceutics, 151:73-80 (2020)). Multiple ratios were tested over 1-5 day incubation times whereby the optimal hydrophobicity from the Log D value was found at day 3 with a charge ratio of 1 siRNA: 0.03 BA (FIG. 10A). siRNA complexes composed of a charge ratio of 1 siRNA: 0.03 BA and siRNA had multiple populations of different sized particles, illustrated by the numerous peaks with z-average sizes of 1840±250 nm in diameter and 2010±300 nm in diameter with PDIs of 0.737±0.29 and 0.686±0.173. 75 nM siRNA BA HIP complexes left at RT for 3 days were incubated in NIH3T3/eGFP cells for 48 hours in the presence of serum containing media resulting in greater than 70% eGFP fluorescence knockdown (FIG. 10C, FIG. 11). This charge ratio is significantly lower than the charge ratio commonly used for nucleic acid delivery using lipid nanoparticles which ranges from 1:1 to 1:3 (Zheng et al., “Lipid Nanoparticle Topology Regulates Endosomal Escape and Delivery of RNA to the Cytoplasm,” Proceedings of the National Academy of Sciences, 120 (27): e2301067120 (2023); Leung et al., “Lipid Nanoparticles Containing SiRNA Synthesized by Microfluidic Mixing Exhibit an Electron-Dense Nanostructured Core,” J. Phys. Chem. C, 116 (34): 18440-18450 (2012)). Commonly, lipofectamine RNAiMax is used as an in vitro transfection agent, thus it was used as our control. Using BA resulted in cytosolic delivery of functional siRNA cargo comparable to the commercially available transfection agent but with no negative effect on cell viability (FIG. 10D). Although it is an effective transfection agent, lipofectamine has known toxicity to many cell lines, because lipofectamine merges with the cell membrane whereas HIP complexes interact with the endosomal membrane (Gigante et al., “Non-Viral Transfection Vectors: Are Hybrid Materials the Way Forward?” Med. Chem. Commun., 10 (10): 1692-1718 (2019). At the current price of $335 per 100 g, BA is 14 million times cheaper per transfection reaction than lipofectamine RNAiMAX and does not require refrigeration. Additionally, HIP complexes can be used with complete media unlike lipofectamine, which requires serum free media.

### Example 5—Less Effective Plasmid DNA Delivery in HeLa and Expi293F™ Significantly Cheaper than Lipofectamine/Expifectamine™

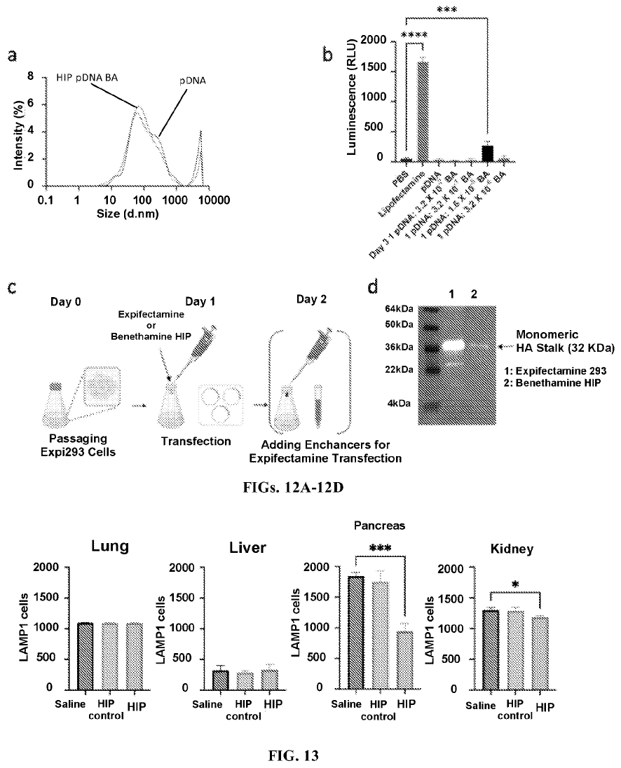

After delivering the double stranded 21-mer siRNA HIP complexes, we examined 5.4 kilo-base-pair (kbp) plasmid DNA (pDNA) delivery by BA HIP. To make HIP complexes, BA was diluted in 1×PBS, then mixed 2-3 times with a pipette with an equal volume of pDNA and used without prolonged incubation (Table 1). HIP complexes were 106±43 nm in diameter while pDNA was 108±25 nm in diameter with PDIs of 0.531±0.17 and 0.531±0.10 respectively (FIG. 12A). The polydispersity was high for pDNA and HIP complexes. To select the optimal amount of counterion for pDNA delivery, 2 μg of a luciferase encoded plasmid was used in Hela cells after 72 hours with these charge ratios: 1 pDNA: 3.2×10⁻⁷ BA, 1 pDNA: 1.6×10⁻⁶ BA, and 1 pDNA: 3.2×10⁻⁶ BA (FIG. 12B). Another group of 1 pDNA: 3.2×10⁻⁷ BA was tested with prolonged incubation where there was no significant luminescence. A charge ratio of 1 pDNA: 1.6×10⁻⁶ BA was the only group which resulted in significant relative luminescence indicating luciferase expression, although much lower than lipofectamine. This charge ratio is significantly lower than the one used for siRNA delivery however, pDNA is much larger than siRNA and BA is not well dissolved ≥30 mg/mL—a solubility limit was reached. To analyze the biomanufacturing potential of HIP pDNA delivery, mammalian and bacterial protein expression were performed. Bacterial protein expression using a plasmid encoding sfGFP(−10) with the same charge ratio above with BA in BL21* produced no colonies after overnight incubation at 37° C., demonstrating the specificity of HIP pDNA delivery to mammalian cells (FIG. 13). Bacteria cells have different membrane structure and function than mammalian cells which undergo endocytosis. Mammalian protein expression of purified hemagglutinin (HA) stalk, used to develop universal flu vaccines, following transfection of Expi293F™ cells was used as a readout (Deng et al., “Double-Layered Protein Nanoparticles Induce Broad Protection against Divergent Influenza A Viruses,” Nature Communications, 9 (1): 359 (2018)). Compared to Expifectamine™ at the same pDNA dose of 50 μg after 5 days, pDNA HIP complexes were not as effective (FIGS. 12C and 12D). However, HIP was still 6,756 times cheaper than ExpiFectamine™ when normalized for expression output, can be dosed in the presence of serum, and does not require refrigeration.

### Example 6—Animal Data Shows HIP Has Therapeutic Potential for siRNA Delivery using Intraperitoneal (IP) Route

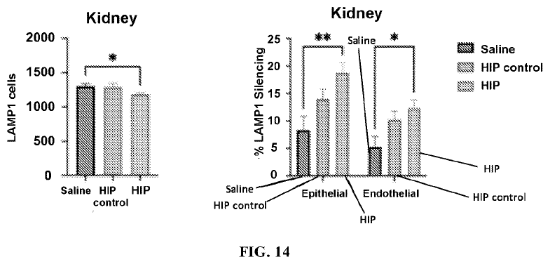

In order to analyze HIP complex delivery for in vivo use, LAMP1 siRNA was selected as LAMP1 is ubiquitously expressed across cell types. LAMP1 silencing does not influence the health or development of mice after 4-6 weeks of age (Da Silva et al., “Universal Barcoding Predicts In Vivo ApoE-Independent Lipid Nanoparticle Delivery,” Nano Lett., 22 (12): 4822-4830 (2022). By injecting a 1.5 mg/kg dose of LAMP1 siRNA using the same charge ratio as in previous siRNA BA HIP delivery, the functional biodistribution was analyzed using flow cytometry and antibody labeling. There was significant gene silencing in the pancreas (~50%) and kidneys (~10%) (FIG. 13). Within the kidneys, there was significant gene silencing in both epithelial and endothelial cell types (FIG. 14).

### Example 7—Discussion of Examples 2-6

Two anionic counterions were tested with cationic protein cargoes, oleic acid (OA) and sodium docusate (SD). OA is a fatty acid and common ingredient in cholesterol supplements that has been used to improve the encapsulation efficiency of insulin, polymyxin B, and others in nanoparticle delivery systems (Ristroph and Prud′homme, “Hydrophobic Ion Pairing: Encapsulating Small Molecules, Peptides, and Proteins into Nanocarriers,” Nanoscale Adv., 1 (11): 4207-4237 (2019); Kozuch, et al., “Insights into Hydrophobic Ion Pairing from Molecular Simulation and Experiment,” ACS Nano, 14 (5): 6097-6106 (2020); Sun et al., “PH-Sensitive Poly(Lactide-Co-Glycolide) Nanoparticle Composite Microcapsules for Oral Delivery of Insulin,” Int J Nanomedicine, 10:3489-3498 (2015); Ristroph et al., “Internal Liquid Crystal Structures in Nanocarriers Containing Drug Hydrophobic Ion Pairs Dictate Drug Release,” J. Colloid Interface Sci., 582:815-824 (2021)). Additionally, OA acts as a permeation enhancer for transdermal nanoparticle delivery by reducing skin barrier function and improving permeation of drugs. FDA approved drugs that use OA to improve skin permeation include celecoxib, adapalene, and tranilast for the treatment of pain, acne, keloids, and other conditions (Atef et al., “Exploring the Potential of Oleic Acid in Nanotechnology-Mediated Dermal Drug Delivery: An up-to-Date Review,” J. Drug Del. Sci. Tech., 67:103032 (2022); Yener et al., “Effect of Vehicles and Penetration Enhancers on the in Vitro Percutaneous Absorption of Celecoxib through Human Skin,” Pharmazie, 58 (5): 330-333 (2003); Salimi et al., “Increase Adapalene Delivery Using Chemical and Herbal Enhancers,” J Cosmet Dermatol, 20 (9): 3011-3017 (2021); Murakami et al., “Topical Delivery of Keloid Therapeutic Drug, Tranilast, by Combined Use of Oleic Acid and Propylene Glycol as a Penetration Enhancer: Evaluation by Skin Microdialysis in Rats,” J Pharm Pharmacol, 50 (1): 49-54 (1998)). SD is the most commonly used ingredient in laxatives. It is the second most common anionic counterion used behind sodium dodecyl sulfate for improved nanoparticle encapsulation, but has less safety concerns and is a highly effective counterion for increased encapsulation efficiency (Bozkir and Devrim, “Design and Evaluation of Hydrophobic Ion-Pairing Complexation of Lysozyme with Sodium Dodecyl Sulfate for Improved Encapsulation of Hydrophilic Peptides/Proteins by Lipid/Polymer Hybrid Nanoparticles,” J Nanomed Nanotechnol, 6 (1): 259 (2015); Griesser et al., “Hydrophobic Ion Pairing: Key to Highly Payloaded Self-Emulsifying Peptide Drug Delivery Systems,” International Journal of Pharmaceutics, 520 (1): 267-274 (2017)). A positive charge variant of super folder green fluorescent protein (sfGFP(+10)), with surface potential of +11.4 at pH 7.4, was also used as a model protein cargo (Dautel and Champion, “Protein Vesicles Self-Assembled from Functional Globular Proteins with Different Charge and Size,” Biomacromolecules, 22 (1): 116-125 (2021)). To evaluate functional protein delivery, OA and SD were paired with positively charged cytochrome C (CytC) since it induces apoptosis upon delivery to the mitochondrial membrane (Bushnell et al., “High-Resolution Three-Dimensional Structure of Horse Heart Cytochrome c,” Journal of Molecular Biology, 214 (2): 585-595 (1990); Eleftheriadis et al., “Cytochrome c as a Potentially Clinical Useful Marker of Mitochondrial and Cellular Damage,” Frontiers in Immunology, 7:279 (2016)).

A cationic counterion, benethamine (BA), was evaluated to deliver negatively charged protein, siRNA, and pDNA into the cytosol. BA is a lipophilic amine, used as a salt in the FDA approved formulation of long acting penicillin G. BA has also been used to improve encapsulation efficiency of retinoic acid in nanoparticles, leading to greater anti-cancer activity than two other cationic counterions, and is non-toxic to cancer cell lines without retinoic acid. Cationic lipids usually have significant toxicity because the hydrophilic headgroup activates pro-apoptotic pathways and pro-inflammatory cascades in a structure dependent manner (Cui et al., “Correlation of the Cytotoxic Effects of Cationic Lipids with Their Headgroups,” Toxicol Res (Camb), 7 (3): 473-479 (2018)). BA, however, lacks a headgroup entirely. A negative charge variant of super folder green fluorescent protein (sfGFP(−10)), with surface potential at pH 7.4 of −9.4, was used as a model protein cargo for pairing with BA (FIG. 2) (Dautel and Champion, “Protein Vesicles Self-Assembled from Functional Globular Proteins with Different Charge and Size,” Biomacromolecules, 22 (1): 116-125 (2021)). Enhanced GFP (eGFP) targeting small interfering RNA (siRNA) was selected as a model small nucleic acid and plasmid DNA (pDNA) coding for influenza hemagglutinin was delivered as model large nucleic acid to test cytosolic and nuclear delivery capability of BA, respectively. Altogether, the combination of different cargos, ions and cell types demonstrate the ability of HIP, achieved by simple mixing of counterion and cargo, to deliver functional biomacromolecules effectively in vitro without a typical nanoparticle carrier.

The results disclosed herein demonstrate that HIP complexes between a charged biomacromolecule and an oppositely charged counterion with only one charge group and no head group making them unique from self-emulsifying drug delivery systems, polyelectrolyte complexes, and lipid nanoparticles. Other systems require more than the two components used in this work. Using incubation of counterion with biomacromolecule we report cytosolic delivery of proteins, siRNA, and pDNA in HeLa, K562, NIH3T3, and Expi293F™ cells. siRNA HIP delivery is as effective as lipofectamine without any impact on cell viability while being 14 million times cheaper. pDNA HIP delivery was not as effective as Expifectamine™; however, was significantly cheaper and does not require a lipid helper on day 2 of transfection, reducing the dedicated time for transfection. pDNA HIP delivery is specific to mammalian cells where bacteria cells yielded no colonies because of the difference in cell membrane structure and function. siRNA delivery was most effective due to its small size and hydrophilicity, and it is hypothesized that this method would work well for similar oligonucleotides such as microRNAs, or these FDA approved oligonucleotides: Patisiran, Givosiran, and Pegaptanib. Herein is demonstrated that HIP complex delivery is tunable for positive and negatively charged biomacromolecules which is applicable for drug discovery, basic research, and therapeutics manufacturing. The cost of this new method is significantly cheaper, does not require refrigeration, does not require RNase free materials, and does not require RNase free water. Additionally, HIP complexes can be treated in the presence of complete media while lipofectamine requires serum free media. By decreasing the cost and toxicity of transfection, other researchers can more efficiently develop nucleic acid and protein therapies and mammalian cell protein expression.

HIP enables functional delivery of siRNA in vivo using simple mixing with non-liver delivery and silencing in the pancreas and kidney from a systemic route of injection. Lipid nanoparticles often result in preferential liver delivery and are unable to achieve non-liver delivery. Transfection reagents like lipofectamine cannot be used in vivo due to toxicity. HIP is functional and a viable option for in vivo screening of siRNA or oligonucleotides in general.

1.-14. (canceled)

15. A method comprising:
mixing a biomacromolecule having a first ionic net charge with a hydrophobic counterion having a second ionic net charge opposite the first ionic net charge under conditions effective to form a complex for intracellular delivery;
wherein the complex is not encapsulated in a nanocarrier.

16. The method of claim 15 further comprising:
delivering the complex to a cell via endocytosis.

17. The method of claim 16 further comprising:
administering the complex to a subject for the delivery of the complex to the cell via endocytosis.

18. The method of claim 15 further comprising:
delivering the complex to an intracellular location of a cell;
wherein:
mixing comprises mixing the biomacromolecule with at least one hydrophobic counterions;
each of the hydrophobic counterions comprises one charge group and no hydrophilic head group; and
delivering the complex to the intracellular location of the cell enables the complex to perform a desired biological function.

19.-21. (canceled)

22. A method comprising:
mixing biomacromolecules having a first ionic net charge with hydrophobic counterions having a second ionic net charge opposite the first ionic net charge under conditions effective to form complexes for intracellular delivery to an intracellular location within a cell that enables the complexes to perform a desired biological function within the cells;
wherein:
none of the complexes are encapsulated in a nanocarrier; and
each of the hydrophobic counterions is selected from a group consisting of oleic acid, sodium docusate, benethamine, arginine hexadecanoyl ester, arginine-nonyl ester, lauramide arginine ethyl ester, 2-naphthalene sulfonic acid, carboxy methyl polyethylene glycol, cholesterol hemisuccinate, decanoic acid, docosahexaenoic acid, linoleic acid, sodium acetate, vitamin E succinate, dodecylamine, maprotiline, N,N-dimethyl dodecylamine, N,N-dimethyl hexylamine, N,N-dimethyl octadecylamine, stearylamine, tetrabutyl ammonium bromide, and triethylamine.

23. The method of claim 22, wherein one or more of:
a molar ratio of the biomacromolecules to the hydrophobic counterions in the complexes is from about 1:10⁻¹⁰ to about 1:396;
a charge ratio of the biomacromolecules and the hydrophobic counterions present in the complexes is from about 1:10⁻¹¹ to about 1:44; or
a mass ratio of the biomacromolecules and the hydrophobic counterions present in the complexes is from about 1:10⁻¹² to about 1:15.

24. (canceled)

25. The method of claim 22, wherein:
a molar ratio of the biomacromolecules to the hydrophobic counterions in the complexes is from about 1:10⁻¹⁰ to about 1:396;
a charge ratio of the biomacromolecules and the hydrophobic counterions present in the complexes is from about 1:10⁻¹¹ to about 1:44; and
a mass ratio of the biomacromolecules and the hydrophobic counterions present in the complexes is from about 1:10⁻¹² to about 1:15.

26. The method of claim 22, wherein the mixing is carried out in the presence of an aqueous buffer.

27. The method of claim 22, wherein:
at least a portion of the biomacromolecules comprise a molecule of cytochrome C; and
at least a portion of the hydrophobic counterions comprise an oleic acid ion.

28. The method of claim 22, wherein;
at least a portion of the biomacromolecules comprise a molecule of cytochrome C; and
at least a portion of the hydrophobic counterions comprise a sodium docusate ion.

29. The method of claim 22, wherein:
at least a portion of the biomacromolecules comprise a molecule of siRNA; and
at least a portion of the hydrophobic counterions comprise a benethamine ion.

30. The method of claim 22, wherein:
at least a portion of the biomacromolecules comprise a molecule of plasmid DNA; and
at least a portion of the hydrophobic counterions comprise a benethamine ion.

31. The method of claim 22, wherein the first ionic net charge has a first magnitude and the second ionic net charge has a second magnitude different than the first magnitude.

32. A method comprising:
administering an intracellular delivery complex to a subject, the intracellular delivery complex comprising:
at least one biomacromolecule having a first ionic net charge; and
at least one hydrophobic counterion having a second ionic net charge opposite the first ionic net charge;
wherein:
the intracellular delivery complex is not encapsulated in a nanocarrier; and
the administering achieves functional intracellular delivery of the intracellular delivery complex to an intracellular location within a cell of the subject that enables the complex to perform a desired biological function within the cell.

33. The method of claim 32, wherein at least one of:
the functional intracellular delivery of the intracellular delivery complex is via endocytosis;
each of the biomacromolecules is a protein, peptide, or nucleic acid; or
each of the hydrophobic counterions comprise one charge group and no hydrophilic head group.

34.-37. (canceled)

38. The method of claim 32, wherein each hydrophobic counterion is selected from a group consisting of oleic acid, sodium docusate, benethamine, arginine hexadecanoyl ester, arginine-nonyl ester, lauramide arginine ethyl ester, 2-naphthalene sulfonic acid, carboxy methyl polyethylene glycol, cholesterol hemisuccinate, decanoic acid, docosahexaenoic acid, linoleic acid, sodium acetate, vitamin E succinate, dodecylamine, maprotiline, N,N-dimethyl dodecylamine, N,N-dimethyl hexylamine, N,N-dimethyl octadecylamine, stearylamine, tetrabutyl ammonium bromide, and triethylamine.

39. The method of claim 32, wherein a molar ratio of the at least one biomacromolecule to the at least one hydrophobic counterion in the intracellular delivery complex is from about 1:10⁻¹⁰ to about 1:396.

40. The method of claim 39, wherein the at least one biomacromolecule and the at least one hydrophobic counterion are present in the intracellular delivery complex in a charge ratio of from about 1:10⁻¹¹ to about 1:44.

41. The method of claim 40, wherein the at least one biomacromolecule and the at least one hydrophobic counterion are present in the intracellular delivery complex in a mass ratio of from about 1:10⁻¹² to about 1:15.

42. The method of claim 32, wherein:
the at least one biomacromolecule comprises a molecule of cytochrome C and the at least one hydrophobic counterion comprises an oleic acid ion;
the at least one biomacromolecule comprises a molecule of cytochrome C and the at least one hydrophobic counterion comprises a sodium docusate ion;
the at least one biomacromolecule comprises a molecule of siRNA and the at least one hydrophobic counterion comprises a benethamine ion; or
the at least one biomacromolecule comprises a molecule of plasmid DNA and the at least one hydrophobic counterion comprises a benethamine ion.

43.-46. (canceled)
